(19) United States
(12) Patent Application Publication
(10) Pub. No.: US 2012/0283510 A1
(43) Pub. Date: Nov. 8, 2012

(54) Pelvic Implants having Perimeter Imaging Features

(75) Inventor: James A. Alexander, Excelsior, MN (US)

(73) Assignee: AMS RESEARCH CORPORATION, Minnetonka, MN (US)

(21) Appl. No.: 13/520,029

(22) PCT Filed: Dec. 29, 2010
(86) PCT No.: PCT/US10/62304

§ 371 Date: Jun. 29, 2012

Related U.S. Application Data
(60) Provisional application No. 61/291,380, filed on Dec. 31, 2009.

(51) Int. Cl. A61F 2/08

(52) U.S. Cl. 600/37

(57) ABSTRACT

An implant adapted to treat various pelvic disorders can include one or more imaging features constructed of a material adapted to provide visualization on an imaging machine (e.g., X-Ray, fluoroscopy, etc.). The imaging feature generally follows along the perimeter shape of at least a portion of the implant to visually confirm how the implant is lying in a patient's body during implantation. The imaging feature can be constructed of a wire (e.g., platinum-iridium), a radio-opaque material or substance, or like materials or constructs adapted to provide the desired visualization.

### PRIORITY

This Application claims priority to and the benefit of U.S. Provisional Application No. 61/291,380, filed Dec. 31, 2009, which is incorporated herein by reference in its entirety.

### FIELD OF THE INVENTION

The present invention relates generally to surgical methods and apparatus and, more specifically, to surgically implantable mesh or sling devices and methods for forming and using the same.

### BACKGROUND OF THE INVENTION

Pelvic health for men and women is a medical area of increasing importance, at least in part due to an aging population. Examples of common pelvic ailments include incontinence (e.g., fecal and urinary), pelvic tissue prolapse (e.g., female vaginal prolapse), and conditions of the pelvic floor.

Urinary incontinence can further be classified as including different types, such as stress urinary incontinence (SUI), urge urinary incontinence, mixed urinary incontinence, among others. Other pelvic floor disorders include cystocele, rectocele, enterocele, and prolapse such as anal, uterine and vaginal vault prolapse. A cystocele is a hernia of the bladder, usually into the vagina and introitus. Pelvic disorders such as these can result from weakness or damage to normal pelvic support systems.

Urinary incontinence can be characterized by the loss or diminution in the ability to maintain the urethral sphincter closed as the bladder fills with urine. Male or female stress urinary incontinence (SUI) generally occurs when the patient is physically stressed.

In its severest forms, vaginal vault prolapse can result in the distension of the vaginal apex outside of the vagina. An enterocele is a vaginal hernia in which the peritoneal sac containing a portion of the small bowel extends into the rectovaginal space. Vaginal vault prolapse and enterocele represent challenging forms of pelvic disorders for surgeons.

Urinary incontinence can be characterized by the loss or diminution in the ability to maintain the urethral sphincter closed as the bladder fills with urine. Male or female stress urinary incontinence (SUI) occurs when the patient is physically stressed.

When any mesh is currently implanted (incontinence, prolapse or the like), it can be very difficult to impossible to determine how the mesh is laying within the body. For example, the physician may want to know if the mesh is lying flat, or if there are bends or bunches in the mesh. Therefore, there is a desire to provide a mechanism, feature or method to determine how the mesh is lying in the body using standard imaging techniques and devices.

### SUMMARY OF THE INVENTION

Disclosed is a feature and method to allow visual confirmation of how the mesh of an implant is lying in the body of the patient. A small platinum iridium wire, or a like structure or marker, is provided around the perimeter of the mesh. This creates a small visible line that can be seen under X-ray, fluoroscopy or like visual imaging means. As a result, the doctor can see if the mesh is flat, or if there are any bends, bunches, or general disruptions in the mesh. This will allow the physician to properly lay the mesh along its proper plane and orientation, thereby eliminating opportunities to damage tissue or reduce the effectiveness of the support mesh.

Various embodiments of the imaging feature can be woven along, molded to, or otherwise disposed along the perimeter of at least a portion of the implant to facilitate imaging. In those embodiments employing a wire or like imaging member, the wire can provide a level of increased rigidity at or near the edge of the implant to facilitate stability and to reduce bunching along the edge or perimeter of the implant.

### BRIEF DESCRIPTION OF THE DRAWINGS

FIG. 1 is a top view of an elongate support implant having one or more perimeter imaging features in accordance with embodiments of the present invention.

FIGS. 2-5 are top views of support implants having arms and a support portion, with one or more perimeter imaging features provided in accordance with embodiments of the present invention.

### DETAILED DESCRIPTION OF PREFERRED EMBODIMENTS

The present invention may be used in conjunction with any mesh or other implant or biologically-compatible graft 10 that is implanted and where the orientation, lay or plane of the implant is desired to be seen with imaging equipment. Examples of such implants 10 are found in implants used to treat pelvic conditions, including incontinence (fecal and urinary) and vaginal prolapse. Various exemplary implants, systems and methods are disclosed in U.S. Pat. Nos. 7,500,945, 7,407,480, 7,351,197, 7,347,812, 7,303,525, 7,025,063, 6,691,711, 6,648,921, and 6,612,977, International Patent Publication Nos. WO 2008/057261 and WO 2007/097994, and U.S. Patent Publication Nos. 2010/0261955, 2002/0151762 and 2002/0147382. Accordingly, the above-identified references are fully incorporated herein by reference in their entirety.

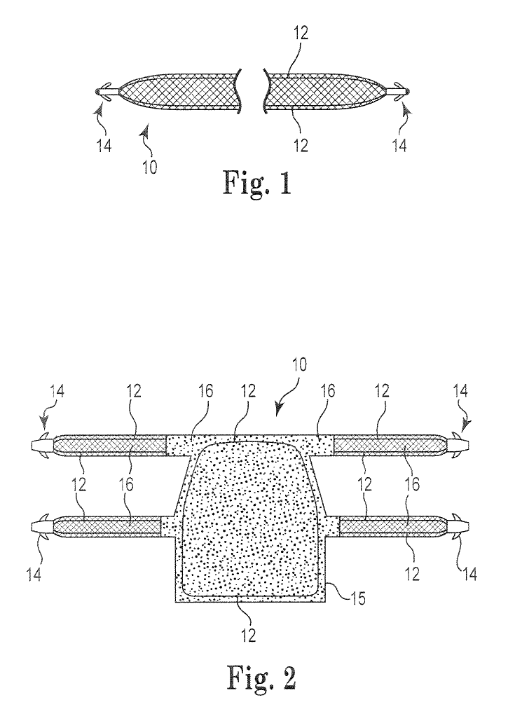

Referring generally to FIGS. 1-5, a distinguishable imaging feature 12 is placed or provided around the perimeter of the implant 10. The imaging feature can include a thin wire 12 material visible to imaging devices, such as X-rays computerized tomography machines, fluoroscopy machines, magnetic resonance imaging machines, or ultrasound machines, to name a few. Other known imaging machines known to one of ordinary skill in the art can be employed to visualize the imaging features as well.

In one embodiment, the wire 12 can be constructed of or include platinum-iridium, or tantalum to facilitate visual imaging to a machine employing or emitting X-rays. The wire 12 can be very thin, such as approximately 0.002 to 0.080 inches in diameter. However, other materials and wire sizes may be selected depending on the application and implant 10 without departing from the scope of the invention. For example, the wire 12 can be smaller than 0.002 inches or greater than 0.080 inches. The wire could be greater than 1 mm. Further, the wire 12 can be integrated into the construct of the implant 10, interwoven with filaments of the mesh implant, or bonded or otherwise provided along a portion of the implant 10. The wire 12 can be generally linear, curved, undulating and the like.

In certain embodiments, the imaging feature 12 can include marking ink, separately formed or differentiated filaments, or like features or indicia not constructed from a separate wire. For instance, radio-opaque ink or other known imaging substances or structures can be added along one or more portions (e.g., filaments or graft material) of the implant 10 to define the imaging feature 12.

As seen in FIG. 1, the implant 10 can be an elongate mesh implant. The mesh implant 10 can be take on a myriad of shapes or sizes depending on the particular application and anatomical requirements for the implant 10. The feature 12, such as an imaging wire, can extend longitudinally along one or more edge portions of the implant 10. The implant 10 can include anchor portions 14. The anchor portions 14 can include the imaging feature 12 in certain embodiments. The feature or wire 12 can extend at or proximate the edge (e.g., longitudinal edge) of the implant 10. However, the feature or wire 12 can be provided or disposed only along defined portions of the implant 10, and is not required to extend along the entire length of the implant 10.

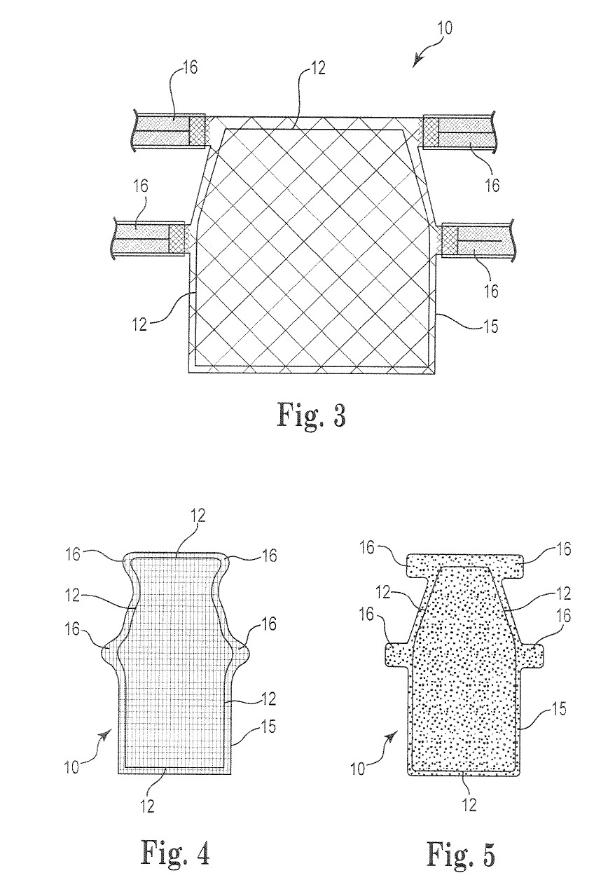

As shown in FIGS. 2-5, the implant 10 can be shaped and sized to treat prolapse or other like pelvic disorders. The implant 10 can include a support portion 15 and one or more extending arms 16. The wire or feature 12 may trace or generally follow the outline of the shape of the implant 10, e.g., the support portion 15. Again, the wire 12 can follow the entire shape, or just a defined or limited portion of the implant 10 perimeter. Further, the wire 12 can extend along the extending arms 16 of the implant 10 for certain embodiments (e.g., FIG. 2). Various anchors 14, mesh portions, sheaths, and like devices, components or structures disclosed in the previously-incorporated references can be employed with the implants 10 of the present invention.

The present invention provides the physician with X-ray assistance (for example fluoroscopy) during implantation, deployment and adjustment. The physician can see if there are any bends, bunches, or general disruptions in the length of the feature or wire 12. If there is any bunching, the wire or feature 12 will display visually as discontinuous or otherwise disrupted, thereby confirming that the implant 10 is not laying flat or in the desired position or orientation. Upon detecting any bunching, the physician can then adjust or correct the plane or positioning of the implant 10 within the patients pelvic region. As such, undesirable bunching or out-of-plane orientation for the implant 10 can be avoided, thereby reducing the chances of erosion or other unwanted tissue irritation or disruption. In certain embodiments, the wire 12 can provide a level of increased rigidity or stiffness along the perimeter or designated portion of the implant 10 to reduce edge bunching or folding.

The implants 10 described herein can be implanted into a patient by use of various different types of surgical tools, including insertion tools, which generally are tools useful to engage and place a tissue anchor or a connector that is secured to an extension portion of an implant. Various types of insertion tools are known, including those in the previously-incorporated references, and these types of tools and modifications thereof can be used according to the present description to install the implants 10.

The implants 10, their various components, structures, features, materials and methods may have a number of suitable configurations and applications, as shown and described in the previously-incorporated references. Various methods and tools for introducing, deploying, anchoring and manipulating implants to treat incontinence and prolapse as disclosed in the previously-incorporated references are envisioned for use with the present invention as well.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

1. A pelvic implant adapted to treat prolapse, comprising:
a support portion;
one or more extending arm portions; and
an imaging wire extending along and generally following the perimeter shape of the support portion, wherein the imaging wire is constructed of a material adapted to provide visualization on an imaging machine.

2. The implant of claim 1, wherein the imaging wire is constructed at least in part of a platinum-iridium material.

3. The implant of claim 1, wherein at least the support portion includes a mesh material.

4. The implant of claim 3, where the mesh material includes a plurality of filaments, and the imaging wire is interwoven with one or more of the filaments.

5. The implant of claim 1, wherein the one or more arm portions include anchoring portions adapted for tissue fixation.

6. The implant of claim 1, wherein the at least the support portion includes a biological graft material.

7. A pelvic implant for treating incontinence, comprising:
an elongate mesh portion having a first longitudinal edge portion, a second longitudinal edge portion, and end portions;
at least one tissue anchor provided at one of the end portions; and
an imaging feature extending along the first and second longitudinal edge portions to generally follow the perimeter edge of the elongate mesh portion, wherein the imaging feature is adapted to provide visualization on an imaging machine.

8. The implant of claim 7, wherein the imaging feature is an imaging wire.

9. The implant of claim 8, wherein the imaging wire is constructed at least in part of a platinum-iridium material.

10. The implant of claim 8, where the elongate mesh material includes a plurality of filaments, and the imaging wire is interwoven with one or more of the filaments.

11. The implant of claim 8, wherein the imaging wire is constructed of multiple separate wires.

12. The implant of claim 7, wherein the imaging feature includes a radio-opaque material.

13. The implant of claim 7, wherein the imaging feature includes a radio-opaque structure.

14. A pelvic implant adapted to treat a pelvic disorder, comprising:
a mesh support portion;
a plurality of arm portions; and
an imaging feature extending along and generally following the perimeter shape of the mesh support portion, wherein the imaging feature is constructed of a material adapted to provide visualization on an imaging machine.

15. The implant of claim 14, wherein the imaging feature is an imaging wire.

16. The implant of claim 15, wherein the imaging wire is constructed at least in part of a platinum-iridium material.

17. The implant of claim 15, where the mesh support material includes a plurality of filaments, and the imaging wire is interwoven with one or more of the filaments.

18. The implant of claim 15, wherein the imaging wire is constructed of multiple separate wires.

19. The implant of claim 14, wherein the imaging feature includes a radio-opaque material.

20. The implant of claim 14, wherein one or more of the arm portions includes an imaging feature constructed of a material adapted to provide visualization on an imaging machine.

21. The implant of claim 20, wherein the imaging feature included with one or more of the arm portions is an imaging wire.
